(19) United States
(12) Patent Application Publication
(10) Pub. No.: US 2004/0073171 A1
(43) Pub. Date: Apr. 15, 2004

(54) Needle-free valve and catheter assembly

(76) Inventors: Bobby E. Rogers, San Diego, CA (US); Mark W. Godfrey, Ramona, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/269,106

(22) Filed: Oct. 10, 2002

(51) Int. Cl. A61M 5/178

(52) U.S. Cl. 604/164.13; 604/167.01

(57) ABSTRACT

An indwelling catheter assembly including a needle-free valve assembly having a valve body, wherein the body has a first end and a second end, the body defines a passageway extending between said first and second ends, and the body defines a sealing surface in the passageway; a guidewire entry port defined by the valve body; a fitting permanently attached to the first end of the needle-free valve assembly; a length of catheter tubing affixed to the fitting; and a stiffening member threaded through the guidewire entry port and disposed within the needle-free valve, wherein the stiffening member extends through the length of catheter tubing is disclosed. Methods of manufacturing the indwelling catheter assembly having a needle-free valve permanently attached thereto are similarly provided. Additionally, methods for preventing the transmission of bloodborne pathogens and nosocomial infections are recited. Furthermore, methods of aspirating fluids or delivering fluids through a catheter assembly having a needle-free valve permanently affixed thereto are provided.

### BACKGROUND OF THE INVENTION

1. Field of the Invention

The invention relates to a safety catheter for intravenous use which is designed to reduce the risk of infection, transmission of bloodborne pathogens, and development of embolisms in a patient. In particular, the invention relates to a needle-free valve fixedly attached to an indwelling catheter such as a peripherally inserted catheter and methods of using the same.

2. Description of the Related Art

In current medical practice, it is commonly necessary to deliver a catheter (a thin flexible tube) into the venous system of a patient to obtain diagnostic information about the heart or its vessels, or to provide therapeutic interventions in certain types of heart conditions. For example, cardiac catheterization can determine pressure and blood flow in the heart's chambers, collect blood samples from the heart, and examine the arteries of the heart by X-ray (fluoroscopy). Fluoroscopy provides immediate (“real time”) visualization of the X-ray images on a screen as well as a permanent record of the procedure.

Catheterization is conducted by placing a needle, cannula, or introducer directly into a patient's vein and threading a catheter through the entry site. Certain indwelling catheters can remain in a patient's venous system for several days, weeks, and even months at a time. Oftentimes, during the course of catheterization, it becomes necessary to deliver fluids such as medications into the patient's venous system at the entry site of the catheter. Additionally, it may be necessary to aspirate fluids from the catheter site to evaluate blood profiles, perform diagnostic assays, etc.

In order to deliver and/or aspirate fluids from the catheter site, the conventional practice has been to establish a vascular access site as described above and then to attach a secondary component (an injection port with a sealed septum) to the catheter to prevent blood leakage, minimize catheter clotting and to facilitate the introduction of medications or aspirate fluids. In this case the introduction or aspiration of fluids is accomplished by utilizing a needle to penetrate the septum thereby accessing the patient's vasculature via the catheter. The needle is generally inserted into a sealed entry port. The main problem with the conventional practice is the necessary use of the second access site as well as the necessary use of a needle. Once a needle has been exposed to a patient's body fluids, it is considered high risk and biohazardous to healthcare workers.

Since the early 1990's, infection by bloodborne pathogens has become a very serious concern to healthcare workers and patients alike. HIV/AIDS and various forms of hepatitis have caused a dramatic shift in the way medical devices are constructed. The elimination of needle sticks and blood contact have been the primary design changes. Many devices are currently marketed as “safety” devices but most fall short of the end goal of preventing the transmission of bloodborne pathogens.

Several devices such as those disclosed in U.S. Pat. Nos. 3,570,484 and 4,324,239 allow access to the patient's bloodstream without the use of a needle. Systems of this type are advantageous in that they eliminate the risk for medical personnel of being stuck by a needle that has been in contact with the patient's body fluid. However, other considerations concern the patient's safety. Namely, any device connected to the patient's bloodstream should not have an exposed fluid reservoir due to the potential for bacterial infection from contact with a tainted object. If the fluid reservoir or cavity is not cleaned, then there is a potential for developing bacteria in the fluid reservoir. The same bacteria could be introduced into a patient's bloodstream when the device is subsequently used to administer new medications or to aspirate fluids from the patient's bloodstream. In some prior approaches to needle-free injection sites, when the injection is complete and the syringe is withdrawn from the injection apparatus so that a partial vacuum is created, blood may be drawn back into the catheter and possibly into the injection apparatus. There is a resulting risk of blood coagulating in the catheter and possibly in the injection apparatus, so that these components must be replaced with possible medical complications as well as additional discomfort, pain, and cost to the patient.

Indwelling Catheters

Turning in particular to the case of indwelling catheters, certain catheters such as Peripherally Inserted Central catheters (“PICCs”), Midline (“Mid”) catheters, and Peripheral catheters can remain in a patient for hours or in some cases as long as 180 days. During the course of treatment with an indwelling catheter, a patient's venous system will be accessed a number of times to either remove or introduce fluids.

The traditional PICC, Mid, or peripheral catheter is constructed of a length of tubing terminating at the proximal end with a female luer hub. The primary difference between these three catheters is the length of the tubing. The rationale for different lengths is driven by the type and duration of the therapy a patient is to receive.

The insertion of these catheters into a patient's venous system can differ procedurally. The peripheral catheter, being the shortest of the three types, is often inserted with a needle disposed within the tubing. After insertion, the needle is removed, leaving the catheter in the vein. Often, blood will flow out of the back of the luer hub and expose the healthcare worker to blood contact. The PICC and Mid are inserted in a similar fashion. Typically, an introducer is placed into the vein first. The introducer is a thin splittable tube whose internal diameter (ID) is closely matched to the outside diameter (OD) of the internally disposed needle in much the same way as described with reference to the peripheral catheter. Once the introducer has been placed in the vein, the needle is removed and again, blood can be seen to come out of the proximal end of the introducer. The introducer will remain in place and acts as a conduit for threading the PICC or Mid into the patient's vein. Once the PICC or Mid is in the vein, the introducer is removed, and blood can often escape from the hub of the catheter. It is an object of the invention to eliminate blood seepage out of the proximal end of the catheter hub.

To date, there is an absence of safety devices on the market which reduce or prevent the risk of exposure to bloodborne pathogens to healthcare workers during catheter insertion. It is therefore an object of the invention to provide a true safety device to address the issue of transmission of bloodborne pathogens during the insertion and use of indwelling catheter devices.

It is a further object of the invention to provide less expensive catheter assemblies to reduce the cost of medical care. In today's market, as stated earlier, PICCs can often be in a patient for periods of time up to 180 days. Valves are attached to these catheters to allow for the introduction of drugs, diagnostic agents, etc. Because the valves are detachable, the Center for Disease Control (“CDC”) dictates that these valves must be replaced every 72 hours. Valves typically cost approximately $1.50/each (USD). Over a 180 day period, on average, sixty valves must be replaced at a cost of nearly $90.00 (USD). Accordingly, there is a need for catheter valves which prevent blood backflow and which are not required to be replaced periodically to reduce healthcare costs.

It is an object of the invention to reduce the incidence of transmission of nosocomial infections to a patient undergoing catheterization. Since 1970, the Center for Disease Control (CDC)'s National Nosocomial Infection Surveillance System (NNIS) has been collecting data on the incidence and etiologies of hospital acquired infections. The majority of hospital acquired bloodstream infections are associated with the use of a catheter, particularly central venous catheters. Today, catheter maintenance requires many manipulations of the catheter. For example, every three days, injection caps, valves, and tubing must be replaced. In so doing, the health care worker can inadvertently expose the patient to nosocomial infections caused by bacteria, viruses, and/or fungi. Each and every manipulation that opens the catheter's closed system exposing it to airborne bacteria and touch contamination is a potential source of danger to the patient. Types of organisms that most commonly cause hospital-acquired bloodstream infections include, without limitation, Staphylocooccus aureus, Candida spp., Escherichia coli, Pseudomonas aeruginosa, Acinetobacter calcoaceticus, and Klebsiella pneumoniae. These microorganisms can then infect the patient's bloodstream and, in extreme cases, sepsis and death can result.

It is yet another object of the invention to reduce the risk of an air embolism to the patient. The insertion of a central catheter exposes a patient to the possibility of “sucking” air through that catheter into their bloodstream. This is a risk that can sometimes result in death or serious injury to the patient. Accordingly, there is a need for catheters designed to eliminate the risk of air embolism to the patient.

There is a constant need for improved safety devices which prevent or reduce the risk of transmission of bloodborne pathogens, loss of patient blood, infection, and air embolism to the patient in the course of insertion of catheter devices into a patient's venous system. Additionally, affordable safety devices would be a great benefit to both the patient and medical community.

### SUMMARY OF THE INVENTION

The invention relates to a safety catheter for intravenous use which is designed to reduce the risk of infection, transmission of bloodborne pathogens, blood loss and development of air embolisms in a patient. In particular, the invention relates to a needle-free valve fixedly attached to an indwelling catheter such as a peripherally inserted catheter and methods of using the same. Accordingly, in one aspect of the invention, an indwelling catheter assembly is provided. The catheter assembly includes a needle-free valve assembly having a valve body, wherein the body includes a first end and a second end, the body defines a passageway extending between the first and second ends, and the body defines a sealing surface in the passageway. The catheter assembly further includes a guidewire entry port defined by a needle-free valve assembly and a fitting permanently attached to the first end of the needle-free valve assembly. A length of catheter tubing is affixed to the fitting; and a stiffening member extending through the guidewire entry port and disposed within the needle-free valve are included, wherein the stiffening member extends past the interior of the fitting and through a portion of the length of catheter tubing.

Optionally, the guidewire entry port is located on the surface of the valve body or the guidewire entry port may be disposed within the fluid passageway of the valve assembly or the guidewire entry port may be disposed at any point from the valve distally up to and including the suture wing. Advantageously, the valve assembly is a positive displacement luer activated valve.

In another aspect of the invention, the indwelling catheter assembly may include a detachable T or Y connector.

In yet another aspect of the invention, a method for aspirating fluids from a catheter site while preventing exposure to bloodborne pathogens is disclosed. The method includes providing an indwelling catheter assembly having a needle-free valve permanently attached thereto and a stiffening member; delivering the catheter into the venous system of a patient; removing the stiffening member; and withdrawing fluids from the venous system of the patient through the needle-free valve. Optionally, the needle-free valve is a positive displacement luer activated valve. The indwelling catheter assembly may be a Peripherally Inserted Central Catheter (PICC); Midline catheter (Mid); or Peripheral catheter. The bloodborne pathogen may include hepatitis B, hepatitis C, and HIV.

In still another aspect of the invention, a method of delivering a fluid to an individual via an indwelling catheter is contemplated. The method includes providing an indwelling catheter assembly having a needle-free valve permanently attached thereto and a stiffening member; delivering the catheter into the venous system of a patient; removing the stiffening member; and accessing the needle-free valve and delivering fluids therethrough. Advantageously, the fluid is a drug or diagnostic agent. The needle-free valve may be a positive displacement luer activated valve. Optionally, the method further includes the step of swabbing the needle-free valve with an antiseptic.

A method of reducing the transmission of bloodborne pathogens during the introduction of an indwelling catheter assembly into a patient's venous system is similarly provided. The method includes introducing into the patient's venous system an indwelling catheter assembly, wherein the assembly includes a proximal end and a distal end, a needle-free valve permanently attached at the proximal end of the catheter assembly, and a stiffening member disposed within the needle-free valve; wherein the needle-free valve prevents the seepage of blood from the proximal end of the catheter assembly; and removing the stiffening member from the catheter assembly. The indwelling catheter assembly may be a PICC, Midline, or Peripheral dwelling catheter. The bloodborne pathogen includes hepatitis B, hepatitis C, and HIV. Advantageously, the needle-free valve is a positive displacement luer activated valve.

In yet another aspect of the invention, a method of reducing the potential for development of an air embolism during the placement of an indwelling catheter into the venous system of a patient is recited. The method includes providing an indwelling catheter assembly having a needle-free valve assembly with a valve body, wherein the body comprises a first end and a second end, the body defines a passageway extending between the first and second ends, and the body defines a sealing surface in said passageway, and wherein the needle-free valve prevents air from being drawn into the catheter assembly; a guidewire entry port defined by the needless valve assembly; a fitting permanently attached to the first end of the needle-free valve assembly; a length of catheter tubing affixed to the fitting; and a stiffening member extending within the guidewire entry port and disposed within the needle-free valve, wherein the stiffening member extends past the interior of the fitting and through a portion of the length of catheter tubing; introducing the indwelling catheter assembly into the patient's venous system; and removing the stiffening member from the catheter assembly. The needle-free valve may be a positive displacement luer activated valve. The catheter may be a PICC, Midline, or Peripheral catheter.

A method of reducing the transmission of nosocomial infection during the aspiration of fluids from or delivery of fluids to an indwelling catheter is provided. The method includes providing an indwelling catheter assembly having: a needle-free valve assembly with a valve body, wherein the body comprises a first end and a second end, the body defines a passageway extending between the first and second ends, and the body defines a sealing surface in the passageway; a guidewire entry port extending through a portion of the needle-free valve assembly; a fitting permanently attached to the first end of the needle-free valve assembly; a length of catheter tubing affixed to the fitting; a luer fitting on the second end of the valve body; and a stiffening member extending within the guidewire entry port and disposed within the needle-free valve, wherein the stiffening member extends past the interior of the fitting and through a portion of the length of catheter tubing; introducing the indwelling catheter assembly into the patient's venous system; removing the stiffening member from the catheter assembly; swabbing the needle-free valve assembly with an antiseptic agent; and withdrawing fluid from the venous system of the patient through the valve or delivering fluid through the needle-free valve to the patient's venous system. The nosocomial infection may be Staphylocooccus aureus, Candida spp., Escherichia coli, Pseudomonas aeruginosa, Acinetobacter calcoaceticus, or Klebsiella pneumoniae. The indwelling catheter may be a PICC, Midline, or Peripheral catheter. Advantageously, the needle-free valve is a positive displacement luer activated valve. Optionally, the guidewire entry port is located on the surface of the valve body or is disposed within the fluid passageway of the valve assembly. The indwelling catheter assembly may further include a detachable T or Y connector.

In another aspect of the invention, a method of manufacturing an indwelling catheter assembly having a needle-free valve permanently attached thereto and a stiffening member, is contemplated. The method includes providing a needle-free valve assembly having a valve body, wherein the body has a first end and a second end, the body defines a passageway extending between the first and second ends, and the body defines a sealing surface in the passageway; locating a guidewire entry port on the surface of the valve body; permanently attaching a fitting to the first end of the needle-free valve assembly; affixing the fitting to a length of catheter tubing; a luer fitting affixed to the second end of the valve body; and extending a stiffening member through the guidewire entry port, past the needle-free valve, and into the interior of a portion of the catheter tubing. The method may further include removeably attaching a T connector or Y connector to the second end of the valve body.

Advantageously, the catheter tubing is constructed from an elastomeric material such as PolyEther Block Amide, PolyOlefins (LDPE, LLDPE, HDPE, PP), PolyStyrene PolyVinyl Chloride (PVC), PolyVinylidene Fluoride (PVDF), Styrene-Ethylene-Butylene-Styrene Block Copolymer, or Thermoplastic PolyUrethanes (TPUs). The valve assembly may be constructed from molded plastic such as ABS, Acrylic Nylons (Nylon 6, Nylon 6/6, Nylon 11, Nylon 12), PolyCarbonate, and Polyester. Optionally, the valve assembly is a positive displacement luer activated valve. Advantageously, the indwelling catheter is a PICC, Midline, or Peripheral catheter.

In yet another aspect of the invention, an indwelling catheter assembly is provided having a needle-free valve assembly with a valve body, wherein the body comprises a first end and a second end, the body defines a passageway extending between the first and second ends, the said body defines a sealing surface in the passageway; a stiffening means for increasing fluid flow area within the catheter assembly and increasing robustness of the catheter assembly; means for extending said stiffening means through the needle-free valve assembly; catheter tubing; and means for permanently attaching the first end of said needle-free valve assembly to said catheter tubing.

### BRIEF DESCRIPTION OF THE DRAWINGS

FIG. 1 is a perspective view of a catheter assembly having a valve integrally attached thereto.

FIG. 2 is a cross-sectional view taken from the proximal end to the distal end of the catheter assembly of FIG. 1 and illustrating one embodiment of a needle-free valve assembly in a first position.

FIG. 3 is a cross-sectional view taken longitudinally from the proximal end to the distal end of the catheter assembly of FIG. 1 and illustrating the valve assembly of FIG. 2 in the second position

FIG. 4 cross-sectional view taken longitudinally from the proximal end to the distal end of the catheter assembly of FIG. 1 and illustrating a needle-free valve assembly in a first position.

FIG. 5 is a cross-sectional view taken longitudinally from the proximal end to the distal end of the catheter assembly of FIG. 1 and illustrating the needle-free valve assembly of FIG. 4 in the second position.

FIG. 6 is a perspective view of a needle-free valve assembly fixedly attached to a catheter having a guidewire extending therethrough.

FIG. 7 is a cross-sectional view of a catheter assembly integrally attached to a valve having a T connector removeably attached thereto.

### DETAILED DESCRIPTION OF THE INVENTION

The invention is directed to catheter assemblies having a valve integrally attached to the catheter and additionally comprising a stiffening wire. The invention relates to a safety catheter for intravenous use which is designed to reduce the risk of infection, transmission of bloodborne pathogens, loss of blood, and development of embolisms in a patient. In particular, the invention relates to a needle-free valve fixedly attached to an indwelling catheter and methods of using the same.

Embodiments of the invention will now be described with reference to the accompanying Figures, wherein like numerals refer to like elements throughout. The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

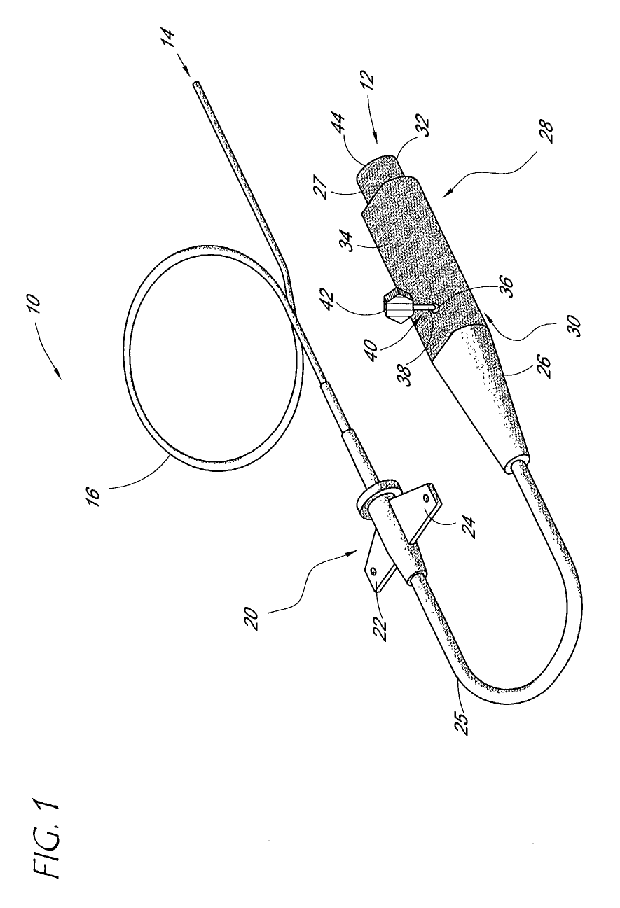

FIG. 1 is a perspective view of a catheter assembly 10 having a proximal end 12 and a distal end 14. The catheter assembly 10 includes catheter tubing 16 having a length “L” at the distal end 14 of the catheter assembly. As described above, the length “L” of the catheter tubing 16 may vary. The length “L” of the tubing 16 is driven by the type and duration of the therapy a patient is to receive. The catheter tubing 16 defines a fluid passageway. The catheter tubing 16 is preferably constructed from a biocompatible thermoplastic material such as the tubing commercially available from, for example, ExtruMed, Inc. (Placentia, Calif.) or Advanced Polymers (Salem, N.H.). The catheter tubing 16 can include coextruded tubing, irradiated high strength balloon tubing, polyolefin heat shrink tubing, single or multi striped tubing, straight or tapered tubing as well as single or multi lumen tubing. In the case of a multi-lumen catheter, multiple needle-free valves can be attached. In preferred embodiments, the multi-lumen tubing would be split into separate lumens at the suture wing 20 (as will be described below) with each lumen terminating with a valve. One of skill in the art will appreciate that the tubing 16 can be constructed from any suitable flexible material such as PolyEther Block Amide (PEBAX®) (available from Modified Polymer Components, Inc. of Sunnyvale, Calif.), PolyOlefins (LDPE, LLDPE, HDPE, PP), PolyVinyl Chloride (PVC), PolyVinylidene Fluoride (PVDF), Styrene-Ethylene-Butylene-Styrene Block Copolymer (C-Flex®) (Consolidated Polymer Technologies, Inc., Clearwater, Fla.), and Thermoplastic PolyUrethanes (TPUs). In a preferred embodiment, the catheter tubing 16 is constructed from Thermoplastic PolyUrethanes (TPUs)

Referring again to FIG. 1, optionally, a pair of suture wings 20 are positioned over and glued, welded, or otherwise permanently affixed to the catheter assembly 10 at the proximal end of the catheter tubing 16. The catheter tubing 16 extends through the suture wings and is adapted for insertion into the venous system of a patient. Extension tubing 25 is located at the proximal end of the suture wings 20. Notably, the extension tubing 25, unlike the catheter tubing 16, is not introduced into the venous system of the patient but rather, remains on the exterior surface of the patient's skin. The extension tubing 25 may or may not be larger in diameter then the catheter tubing 16. The extension tubing 25 may or may not be an extension of the catheter tubing 16 through the suture wing 20. Often at the valve 28 or suture wing 20 (wherever the catheter tubing ends), there may be another larger diameter tube over the catheter material to act as a strain relief. In one embodiment, the distal end of the suture wings 20 is bonded to the proximal end of the catheter 10 and then extension tubing 25 is glued, welded, compression fit, or otherwise permanently attached to the proximal end of the wings 20 and similarly permanently bonded to the valve assembly 28. The valve assembly 28 will be described in greater detail below. In another embodiment, the extension tubing 25 is co-molded to the suture wing. As illustrated in FIG. 1, the suture wings 20 include a first wing 22 and a second wing 24. The suture wings 20 serve to anchor down the catheter tubing 16 after insertion of the catheter tubing 16 into the venous system of the patient so the tubing 16 cannot be pulled out of the patient or vein. Having movement in and out of the venipuncture site can lead to infection and patient discomfort. The suture wings 20 are used to attach the catheter to the patient via sutures, tape, or some other securement device. In another embodiment, the extension tubing 25 comprises the catheter tubing 16 extended through the suture wing 20 to the valve 28. This section of tubing between suture wings 20 and valve 28 there may or may not be another larger diameter of tubing over the catheter tubing to act as a strain relief.

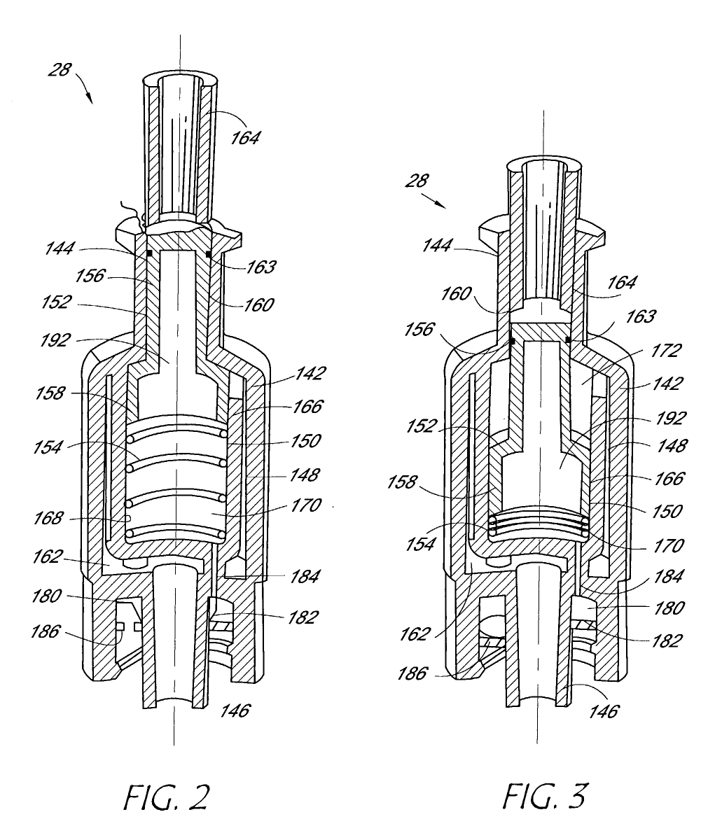

Still with reference to FIG. 1, in some embodiments, a fitting 26 is coupled to the proximal end of the catheter tubing 16 and to the distal end of the valve assembly. The fitting 26 is advantageously constructed of a molded plastic material. As illustrated, the fitting 26 is generally frusto conical in shape. However, it will be appreciated that the shape of the fitting 26 is not a critical feature of the invention. The fitting 26 is designed to enable fluid communication between the catheter tubing 16 and a needle-free valve assembly 28, as will be described in greater detail below with reference to FIGS. 2, 3, and 4. Preferably, the fitting 26 is in the form of a female fitting. One of skill in the art will appreciate that the fitting 26 may be any type of fitting, including a luer fitting. The fitting 26 is used for the permanent attachment by bonding, welding, or compression fit of the catheter tubing 16 or extension tubing 25 to the valve assembly 28.

Integrally attached to the fitting 26 is a needle-free valve assembly 28. As used herein, the phrase “integrally attached” is defined to mean permanently attached, welded, bonded, or adhered to in a fixed manner. One of skill in the art will appreciate that the fitting 26 may not be a separate component of the catheter assembly but rather, the catheter tubing 16 can be permanently attached by bonding, welding, adhering, or compression fit directly to the distal end 30 of the needle free valve assembly 28. The valve assembly 28 includes a proximal end 32 and a distal end 30, and a valve body 34. The valve assembly 28 does not require a needle to administer medication to a patient or to aspirate fluids from the patient. Therefore, the risk of transmission of bloodborne infections to the healthcare worker is greatly reduced. Additionally, the valve assembly 28 is adapted for ease of cleaning (i.e. swabbing with alcohol to keep the catheter site generally sterile and to reduce the risk of transmission of nosocomial infections to the patient). More particularly, the valve assembly 28 of the invention does not have an exposed fluid reservoir that could become a breeding ground for bacteria. Moreover, because the needle-free valve assembly 28 is permanently affixed to the catheter, the catheter assembly is a closed system. Blood is prevented from seeping out of the proximal end of catheter by the valve assembly 28, further minimizing the exposure of health care workers to bloodborne pathogens from a patient. Unlike open catheters which allow air to be sucked into the system and pose a risk for developing an embolism, the catheter assembly of the invention includes a permanently attached valve assembly which prevents air from being pulled into the catheter and therefore reduces the incidence of embolism development. Another feature of the valve assembly 28 is the fact that the valve assembly is permanently attached to the catheter assembly. Because the valve is not detachable, it is not governed by the CDC mandate which dictates that detachable valves must be replaced every 72 hours. Thus, the valve of the invention can remain attached to the catheter for the duration of the treatment process.

It will be appreciated by one of skill in the art that, although certain valve assemblies are described later in FIGS. 2, 3, 4, and 5 as preferred embodiments, any needle-free valve assembly having the foregoing characteristics is contemplated as being suitable for integral attachment to the fitting 26 of the catheter assembly 10 or to be integrally attached to the extension tubing 25 of the catheter assembly 10. For example, the needle-free valves described in U.S. Pat. Nos. 6,029,946; 6,168,137; 6,158,458; 6,146,362; 6,117,114; 6,068,617; 6,063,062; and 6,050,978, hereby incorporated by reference, can be permanently affixed to the proximal end of a fitting 26 or directly to extension tubing 25 without deviating from the spirit of the invention. Likewise, the valves disclosed in U.S. Pat. Nos. 6,029,946; 5,954,313; 5,921,264; 5,833,674; 5,788,215; 5,674,206; 5,654,538; and 5,540,661, hereby incorporated by reference, may be used to carry out the invention. The advantages of having a needle-free valve fixedly attached to a catheter assembly are numerous. Such a valve can be used repeatedly without the use of a needle to introduce medications into, or aspirate fluids from, a patient's blood. Advantageously, the valve assembly includes an easily cleanable top surface to reduce the possibility of bacterial infection by repeated use of the valve assembly 28.

Distal to the inlet port 44, a guidewire entry port 36 on the surface of the valve body 34 is provided for entry of a stiffening member 38. The stiffening member is configured for insertion through the guidewire entry port 36, past the distal end of the valve 30, and into the catheter tubing 16. In another embodiment, the guidewire entry port 36 and stiffening member 38 are positioned axially through the proximal end of the valve assembly 28 as will be described below with reference to FIG. 5. The stiffening member 38 has a proximal end 40 a distal end (not illustrated), and a length “L”. As described with reference to the catheter tubing 16, the length “L” of the stiffening member is dictated by the length of the catheter tubing, which in turn is prescribed by the type and duration of the therapy a patient is to receive. At the proximal end 40 of the stiffening member, a wire handle 42 is provided. The stiffening member 38 adds structural integrity to the catheter tubing, thereby facilitating introduction of the catheter device into the venous system of a patient. Oftentimes, catheter lines are made of a soft bio-compatible material which is very difficult to insert into a patient because its soft or pliable construction causes the catheter tubing to collapse or bend before entering the patient's veins. In order to more quickly and efficiently insert a catheter such as PICC, Mid, or peripheral catheter, a flexible stylet, guidewire, or needle is used as a stiffening member inside the catheter during insertion. With regard to the flexible stylet or guidewire, the stiffening member is an elongated stylet that is disposed within the catheter, such that the stylet can occupy less than half of the lumen area of the catheter. As a result, adequate fluid flow is allowed between the stylet and the inner wall of the catheter. The stiffening member is constructed from a lightweight, flexible, biocompatible metal such as twisted or wound stainless steel with a blunt distal end. In some embodiments, the stiffening member may be treated with a coating such as a hydrogel or Teflon™ or a Teflon™-type coating to minimize friction between the guidewire and the inner wall of the catheter and to reduce the possibility that the guidewire may perforate a catheter when it is forced against the catheter. Once the catheter is placed inside the patient, the stiffening member is removed by pulling outwardly on the wire handle 42. Concurrent with the process of removing the stiffening member 38, a syringe (not illustrated) can be connected to the inlet port 44 of the valve located at the proximal end of the valve body 34 to flush the catheter during wire retraction. In certain embodiments, when the catheter tubing 16 is a multi-lumen tubing, the stiffening member 38 would only be included in one of the lumens.

A luer fitting 27 is located at the proximal end 32 of the valve body. The luer fitting 27 is preferably a female luer fitting suitable for connection with an IV set or syringe. The luer fitting 27 is adapted for the attachment of T connectors, Y connectors, IV sets or syringes. Advantageously, all of these “attaching devices” have a male luer for mating with the luer fitting at the proximal end 32 of the valve body.

In preferred embodiments, the needle-free valve assembly 28 is a positive displacement luer activated valve such as the valves described in U.S. Pat. No. 5,006,114 and co-pending U.S. patent application Ser. Nos. 09/410,419, 09/602,540, and 10/023,195, the entire contents of which are hereby incorporated by reference in their entirety. These valves are designed to prevent blood from being drawn back into the catheter and the valve assembly at the conclusion of the delivery or aspiration of fluids to or from a patient's venous system. Additionally, these valves avoid potential clogging of the catheter or valve from blood drawn back through the catheter, while at the same time avoiding potential contamination of the interior of the valve due to contaminants external to the patient that find their way into the interior of the valve. These valve assemblies are swabbable; that is the valves obviate the need for an external fluid reservoir which can become an area for the growth of bacteria, thereby preventing the introduction of bacteria into the patient's bloodstream upon subsequent use of the device for dispensing medication.

FIGS. 2 and 3 illustrate one embodiment of the needle-free valve assembly 28 of the present invention. As will be described in greater detail below, FIG. 2 is a cross-sectional view of a needle-free valve assembly 28 in a first position while FIG. 3 illustrates a cross-sectional view of a needle-free valve assembly 28 in a second position. Turning now to FIG. 2, the valve assembly 28 includes a hollow housing 142 having an inlet 144, an outlet 146, and a fluid channel 148 extending between the inlet 144 and the outlet 146. The fluid channel 148, positioned between the outer wall 168 of the bore 150 and the inner wall of the housing 142, provides the fluid flow path between the inlet 144 and the outlet 146. The inlet 144 is designed to be readily accessible for cleaning, as with an alcohol swipe, to prevent contamination through the inlet 144. A bore 150 is positioned within the housing 142. The bore 150 is closed at one end, which is preferably the end remote from the inlet 144. In the illustrated embodiment, the bore 150 is substantially cylindrical with a bore axis 151, and the inlet 144 and the outlet 146 are coaxial along the bore axis 151. In an alternative embodiment, the outlet 146 need not be coaxial with the bore axis 151. A piston 152 is slidable within the bore 150 between a first position (as illustrated in FIG. 2) relatively closer to the inlet 144 and a second position (as seen in FIG. 3) relatively farther from the inlet 144. The piston 152 is biased toward the first position by a spring 154, here illustrated as a coil spring.

The piston 152 has a first portion 156 and a second portion 158. The fluid channel 148 has a first length 160 positioned adjacent to and radially outwardly from the first portion 156 of the piston 152, when the piston 152 is in the first position. The first length 160 of the fluid channel 148 controllably communicates with the inlet 144 when the piston 152 is in the second position. A second length 162 of the fluid channel 148 communicates between the first length 160 and the outlet 146. When the piston 152 is forced to the second position, as shown in FIG. 3, by the insertion of an end 164 of a needle-free syringe, the axial movement of the first portion 156 of the piston 152 unseals the first length 160 of the fluid channel 148. The act of unsealing the first length 160 of the fluid channel 148 is accomplished by moving the piston inlet seal 163 below the top of the first length 160 of the fluid channel 148 and allowing injected fluid to flow from the syringe, through the inlet 144, and through the first length 160 and second length 162 of the fluid channel 148. Fluid continues to flow through the outlet 146, and thence to the patient through the catheter assembly 10 that is attached to the outlet 146.

A piston seal 166, which may be a sliding seal, located between the second portion 158 of the piston 152 and a wall 168 of the bore 150, divides the bore 150 into a sealed chamber 170 (below the piston 152 in FIG. 2) and an unsealed chamber 172 (above the piston 152 in FIG. 3). The unsealed chamber 172 serves as a fluid reservoir. In a preferred embodiment, the spring 154 is located within the sealed chamber 170. When the piston 152 is in the second position, a new volume in the form of the unsealed chamber 172 within the bore 150 is created. The valve 28 with the new volume 172 acts as a needle-free valve fluid capacitor.

The unsealed chamber 172 is in communication with the fluid channel 148. When the piston 152 is moved to the second position shown in FIG. 3 for sterile fluid injection, a small amount of the fluid flows into the unsealed chamber 172. At the conclusion of the fluid injection as the end 164 of the syringe is withdrawn so that the piston 152 moves back toward the first position shown in FIG. 2, the fluid in the unsealed chamber 172 is expelled into the fluid channel 148. Because the movement of the piston 152 has sealed the inlet 144 from the fluid channel 148 as described above, the fluid expelled from the unsealed chamber 172 flows toward the outlet 146 and thence into the catheter 10 and in the direction toward the vein of a patient. The drawing of blood back into the catheter 10 is thereby prevented.

One concern with this approach is that the pressure in the sealed chamber 170 may become sufficiently large, when the piston is moved to the second position of FIG. 3, that the built-up pressure may interfere with the operation of the piston 152 and consequently, the operation of the valve assembly 28. For this reason, a vent to atmosphere of the sealed chamber 170 so as to avoid the pressure buildup may be provided. However, as discussed above, contamination may find its way from the exterior through the vent, past the piston seal 166, into the unsealed chamber 172, and into the fluid channel 148 where it contaminates the fluid within the valve assembly 28. Undesirably excessive high pressure can be avoided by two primary approaches: in a first of these, a gas volume separated from the exterior of the housing by a balloon membrane serves to receive air expelled from the sealed chamber 170 as the piston moves to the second position; and in the second approach, an internal gas accumulator is used.

A gas volume 180 is defined by a portion of the housing 142 and a balloon membrane 182. The gas volume 180 communicates with the sealed chamber 170 through a port 184. When the piston 152 is in the first position of FIG. 2, the balloon membrane 182 is collapsed and the gas volume 180 is relatively small. When the piston 152 is moved to the second position of FIG. 3, air flows from the sealed chamber 170 through the port 184 and into the gas volume 180, expanding the balloon membrane 182.

A wall 186 is provided to prevent the balloon membrane 182 from inadvertently rupturing or becoming damaged. This balloon membrane approach can be employed wherever there is some available volume within or at the surface of the housing 142. The illustrated embodiment places the gas volume 180 near the outlet 146. In another embodiment, the gas volume may be on the lateral side of the bore 150 so that the balloon membrane 182 expands into the fluid flow channel 148. Alternatively, the gas volume 180 is placed within the housing 142 by removing the housing material to define an available volume. In yet another embodiment, the gas volume 180 is located within the unsealed chamber 172 (with the port 184 being through the piston 152 itself and with care taken to be certain that the balloon membrane 182 does not interfere with the sealing action of the piston 152 to seal the inlet 144), or near the inlet 144. Care is taken so that the balloon membrane 182 does not negate the effect of the unsealed chamber 172 in serving as the fluid capacitor.

In a preferred embodiment, an internal gas accumulator 192 is provided. In the approach illustrated in FIG. 3, the entire interior of the piston 152 is hollowed out to define the gas accumulator 192. Alternatively, gas accumulator volume may be provided within the wall of the housing 142. This approach differs from the balloon membrane approach described above in that there is no need for a membrane in the gas accumulator approach because there is no communication between the sealed chamber 170 and the exterior of the housing 142 or the unsealed chamber 172. By adding a constant accumulator volume to the sealed chamber 170, the maximum value of the pressure experienced in the sealed chamber 170 when the piston 152 is in the second position is reduced, as compared with the maximum pressure experienced in the absence of the accumulator volume.

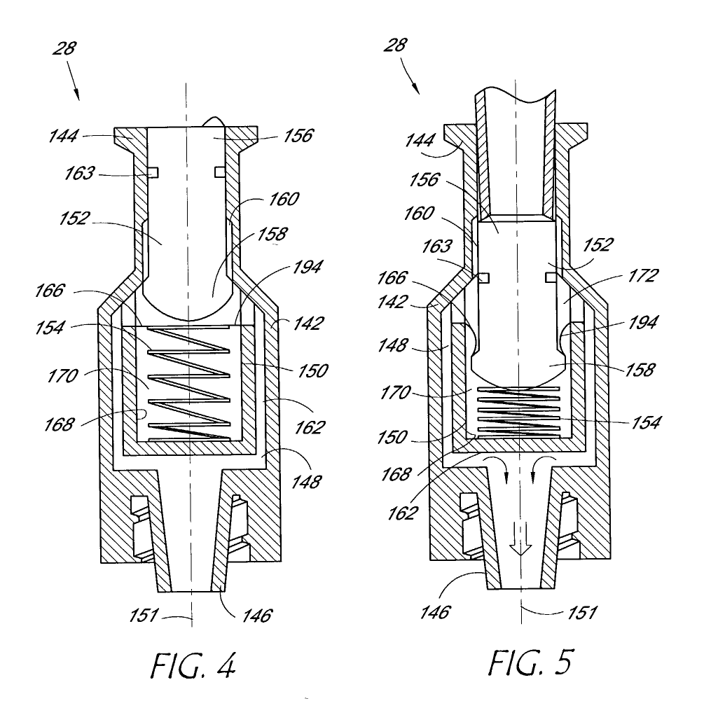

FIGS. 4 and 5 illustrate yet another approach to a needle-free valve assembly suitable for use in the invention. The basic operation of the embodiment of the valve assembly 28 is like that discussed with reference to FIGS. 2 and 3, and the prior description is incorporated here. In this case, the piston seal 166 is a flexible membrane seal 194. As the piston 152, moves from the first position of FIG. 4 toward the second position of FIG. 5 to increase the gas pressure within the sealed chamber 170, the membrane seal 194 bulges in to the unsealed chamber 172 to relieve the pressure within the sealed chamber 170. Care is taken that the membrane seal 194 in its bulged state does not fill the unsealed chamber 172 and negate its fluid capacitor functionality. In another embodiment, the chamber 170 may be vented to atmosphere. This is possible because the membrane seal 194 acts as a barrier preventing contamination of chamber 170 and fluid path 148.

In yet another embodiment, any of these ventless structures may be assembled such that a gas pressure within the sealed chamber 170 is less than a gas pressure within the unsealed chamber 172, when the piston 152 is in its first position. This state may be achieved by assembling the valve assembly 28 and completing the piston seal 146 with the valve assembly 28 in a reduced pressure environment such as a partial vacuum chamber or at high altitude. By reducing the pressure in the sealed chamber 170 with the piston 152 in the first position, the pressure within the sealed chamber 170 with the piston 152 in the second position is not as great as it would be otherwise.

The materials used for manufacturing the needle-free valve assembly 28 should have the characteristics of a currently acceptable medical grade plastic that can be precision molded and adapted to maintain its dimensions under normal hospital conditions. When a positive displacement valve is employed, the valve component material is resistant to alcohol and has a low coefficient of friction. Examples of suitable materials include polycarbonate, PVC, nylon, delrin, and hydrel. In a preferred embodiment, the valve assembly 28 is constructed from polycarbonate because of its long shelf life, ability to be sterilized, and use in a clear, translucent, or colored form. The piston 152 should likewise be manufactured from medical grade plastic which can be precision molded and maintain its dimensions under normal conditions. In a particularly preferred embodiment, the piston 152 is substantially rigid, resistant to alcohol, injection moldable, and low in cost. Suitable materials include valox, polycarbonate, delrin, nylon, etc. In a particularly preferred embodiment, the piston is fabricated from valox.

The seals 163 and 166 on the piston are o-ring seals. The o-rings are constructed of materials that, in combination with the materials of the housing 142, provides a low coefficient of friction and lends itself to a medical grade silicone oil lubricant coating. Suitable materials for the construction of the o-ring seals include EPDM, nylon, delrin, hydrel, polyurethane, silicone, or other thermoplastic elastomeric materials. In a particularly preferred embodiment, the o-rings are fabricated from EPDM.

The spring 154 can be a plastic or metal spring, a high viscosity silicone, air, or a combination of any of these. Elastomeric materials include cellular, noncellular, synthetic rubbers and plastics, such as highly dense or closed polyurethane, styrene butadenes, or isoprenes as described in U.S. Pat. No. 4,324,239, which is hereby incorporated by reference.

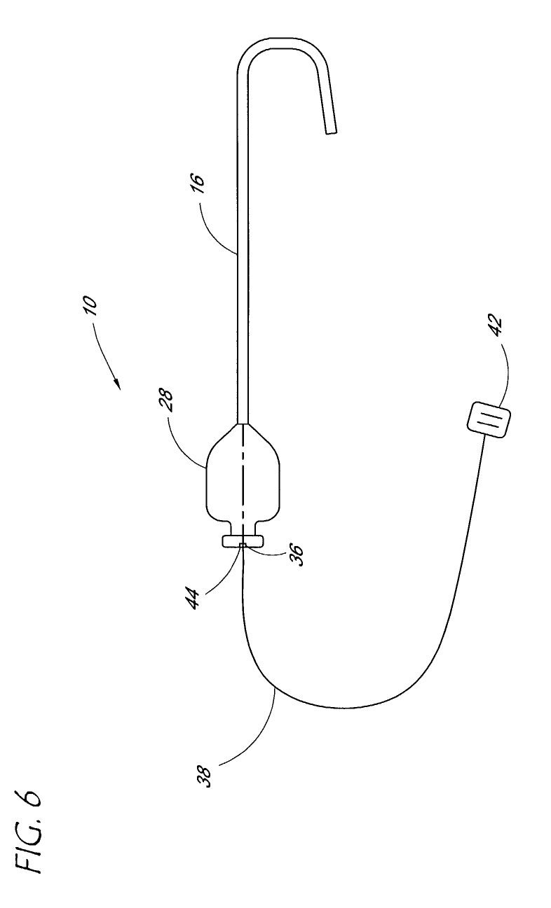

FIG. 6 illustrates an alternate embodiment, wherein a catheter assembly 10 includes a guidewire entry port 36 for the stiffening member 38 located at the proximal end of the valve assembly 28. FIG. 6 is a perspective view of one embodiment of a catheter assembly 10 with a guidewire entry port 36 in the piston of the valve 38. Notably, the precise location of the guidewire entry port 36 is not critical as long as it is within the sealing mechanism of the valve's proximal port and proximal to catheter tubing 16. As illustrated in FIG. 6, the guidewire entry port 36 is positioned in the center of inlet port 44 of the valve assembly 28. However, in another embodiment, the guidewire entry port 36 is slightly offset from the inlet port 44 of the valve 28. Flushing of the system post guidewire removal is easily accomplished in the typical fashion of attaching a syringe to the valve and depressing the plunger.

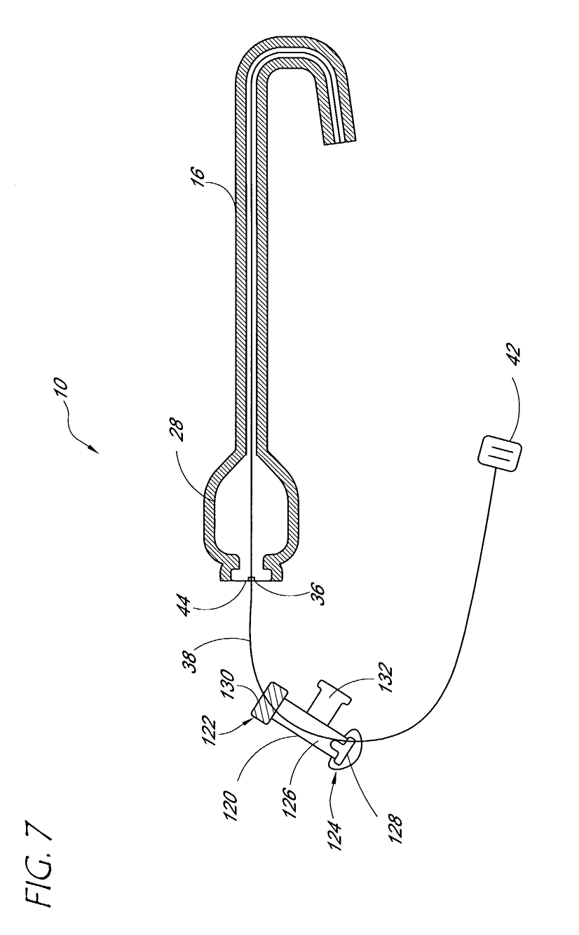

Referring now to FIG. 7, another embodiment of the invention is illustrated, wherein the catheter assembly 10 additionally includes a T connector 120. FIG. 7 is a perspective view of a catheter assembly 10 including a valve 28, stiffening member 38, and a T connector 120 removeably attached to the valve 28. The T connector 120 has a distal end 122 and a proximal end 124 and an axial fluid passageway 126 extending therebetween. The stiffening member 38 is disposed within the T connector 120 and extends into the valve assembly 28 and through the catheter tubing 16 as illustrated. At both the distal 122 and proximal end 124 of the T connector 120, female port 128, and male port 130 are provided. Port 128 is covered with a resealable, self-sealing elastomeric material such as synthetic or natural rubber to seal the port 124. Located between the distal end 122 and proximal end 124 of the T connector 120 there is a female port 132 configured for attaching a syringe (not illustrated) thereto. The T connector 120 is removeably attached to the valve assembly 28 at the fluid inlet port 44. The attachment of the T connector 120 to the valve assembly 28 can be via a luer lock connection or a luer slip configuration. After placement of the catheter assembly 10 into a patient's venous system, the stiffening member 38 is removed by pulling outwardly on the wire handle 42. A syringe can be attached to the T connector 120 at the female port 132 to flush the catheter assembly 10, thereby aiding in the withdrawal of the stiffening member 38. Fluid is flushed from the syringe through the female port 132 of the T connector 120 and into the valve assembly 28 as the stiffening member 38 is retracted. Attaching a syringe to the open port on the T or Y connector allows fluid flow through the now opened valve while the user retracts the wire through the septum. This is done in a pull-flush-pull-flush-pull manner. Once the stiffening member 38 has been removed, the T connector 120 is detached from the valve 28, removed, and discarded. Upon removal, the valve closes, pushing fluid forward by its positive displacement characteristic. Although not illustrated, it will be appreciated that a Y shaped connector such as the one described in U.S. Pat. No. 4,048,995, hereby incorporated by reference, may also be used in place of the T connector to facilitate the removal of the stiffening member.

A method of manufacturing a catheter assembly having a needle-free valve 28 fixedly attached to the catheter 10 is likewise provided. The method includes locating catheter tubing 16 at the distal end 14 of a fitting 26 and attaching the catheter tubing 16 thereto. Attachment can be accomplished by bonding, gluing, ultrasonically welding, or a compression fitting of the tubing to the fitting. At the proximal end of the fitting, a needle-free valve assembly 28 having a guidewire entry port 36 is permanently attached. A stiffening member 38 is threaded through the guidewire entry port 36. Optionally, a removable T or Y connector 120 is connected to the proximal end of the needle-free valve assembly 28.

In another embodiment, a method of delivering or aspirating fluid to or from a patient via an indwelling catheter assembly is provided. The method includes introducing an indwelling catheter 10 having a needle-free valve 28 fixedly attached to the catheter assembly 10 into the venous system of a patient. Once in place, fluids can be delivered to a patient and/or fluids can be removed from a patient from the needle-free valve 28 as was described with reference to FIG. 4.

The invention provides a method of reducing the transmission of bloodborne infections from patients to healthcare workers. Once a catheter is placed within a patient's venous system, blood is often seen seeping out the proximal end of the catheter. By fixedly attaching a needle-free valve assembly to the proximal end of a catheter assembly, blood is blocked from exiting the proximal end of the catheter. Accordingly, the healthcare worker inserting the catheter assembly is protected from exposure to patient blood. Additionally, because the valve assemblies are needle-free valves, when fluid such as medication is delivered through the valve or when fluid is aspirated from the valve, the healthcare worker is not exposed to needles or the dangers posed by needle sticks. Thus, the risk of transmission of bloodborne pathogens such as HIV, various strains of hepatitis, etc. is reduced or eliminated.

Similarly, a method of reducing the risk of the transmission of infection to the patient is contemplated by the invention. Accordingly, in one embodiment of the invention, a method of reducing transmission of infectious agents into the vascular system of a patient in the course of placement of an indwelling catheter device is provided. By utilizing a catheter assembly having a needle-free valve permanently attached thereto, blood is prevented from exiting the catheter. In addition, unneeded connect/disconnect manipulations of valves and tubing to the catheter are eliminated. As such, a patient's blood is not subject to increased exposure to nosocomial infection at the catheter's proximal end. Moreover, because the valve is swabbable, alcohol or other antiseptics can be used to create a sterile environment at the valve site and the incidence of transmission of nosocomial infections to a patient at the catheter site is greatly reduced.

In another embodiment, a method of reducing patient blood loss during the placement of a vascular device into a patient's venous system is provided. Typically, when vascular devices such as indwelling catheters or diagnostic catheters are placed in a patient's venous system, blood will seep out of the proximal end of the catheter upon removal of the introducer. By employing a catheter assembly having a valve permanently affixed to the proximal end of the catheter, blood loss is minimized. The valve prevents the backflow of blood out of the catheter. Accordingly, the blood stays in the patient and does not flow unabated out of the catheter.

In addition to preventing the transmission of blood borne-infections, the invention contemplates methods of reducing the development of air emboli attendant with the placement of a catheter or other vascular device into the venous system of a patient. In some cases, an open catheter can allow air to be sucked into the patient, threatening the patient with an air embolism. However, by utilizing a catheter assembly having a needle-free valve attached to the catheter to prevent blood backflow, the incidence of air emboli is greatly diminished because the catheter remains closed.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. An indwelling catheter assembly, comprising:
a needle-free valve assembly having a valve body, wherein said body comprises a first end and a second end, said body defines a passageway extending between said first and second ends, and said body defines a sealing surface in said passageway;
a guidewire entry port defined by the needle-free valve assembly;
a fitting attached to said first end of said needle-free valve assembly;
a length of catheter tubing affixed to said fitting; and
a stiffening member extending through said guidewire entry port and disposed within said needle-free valve assembly, wherein said stiffening member extends past the interior of said fitting and through a portion of the length of catheter tubing.

2. The indwelling catheter assembly of claim 1, wherein said fitting is permanently attached to said first end of said needle-free valve assembly.

3. The indwelling catheter assembly of claim 1, wherein said guidewire entry port is located on the surface of said valve body.

4. The indwelling catheter assembly of claim 1, wherein said guidewire entry port is located on the surface of said fitting.

5. The indwelling catheter assembly of claim 1, wherein said guidewire entry port is disposed within said fluid passageway of said valve assembly.

6. The indwelling catheter assembly of claim 1, wherein said valve assembly is a positive displacement luer activated valve.

7. The indwelling catheter assembly of claim 1, further comprising a detachable T connector.

8. The indwelling catheter assembly of claim 1, further comprising a detachable Y connector.

9. A method of aspirating fluids from a catheter site while preventing exposure to bloodborne pathogens, comprising:
providing an indwelling catheter assembly having a needle-free valve permanently attached thereto and a stiffening member;
delivering said catheter into the venous system of a patient;
removing said stiffening member; and
withdrawing fluids from the venous system of said patient through said needle-free valve.

10. The method of claim 9, wherein said needle-free valve is a positive displacement luer activated valve.

11. The method of claim 9, wherein said indwelling catheter assembly is selected from the group consisting of a Peripherally Inserted Central Catheter (PICC); Midline catheter (Mid); and Peripheral catheter.

12. The method of claim 9, wherein said bloodborne pathogen is selected from the group consisting of hepatitis B, hepatitis C, and HIV.

13. A method of delivering a fluid to an individual via an indwelling catheter, comprising:
providing an indwelling catheter assembly having a needle-free valve permanently attached thereto and a stiffening member;
delivering said catheter into the venous system of a patient;
removing said stiffening member; and
accessing said needle-free valve and delivering fluids therethrough.

14. The method of claim 13, wherein said fluid is a drug.

15. The method of claim 13, wherein said fluid is a diagnostic agent.

16. The method of claim 13 wherein said needle-free valve is a positive displacement luer activated valve.

17. The method of claim 13, further comprising the act of swabbing said needle-free valve with an antiseptic.

18. The method of claim 13, wherein said indwelling catheter assembly is selected from the group consisting of a Peripherally Inserted Central Catheter (PICC); Midline catheter (Mid); and Peripheral catheter.

19. A method of reducing the transmission of bloodborne pathogens during the introduction of an indwelling catheter assembly into a patient's venous system comprising:
introducing into the patient's venous system an indwelling catheter assembly, said assembly comprising a proximal end and a distal end, a needle-free valve permanently attached at the distal end of said catheter assembly, and a stiffening member disposed within said needle-free valve into the patient's venous system; wherein said needle-free valve prevents the seepage of blood from the proximal end of the catheter assembly; and
removing said stiffening member from said catheter assembly.

20. The method of claim 19, wherein said indwelling catheter assembly is selected from the group consisting of a PICC, Midline, and Peripheral dwelling catheter.

21. The method of claim 19, wherein said bloodborne pathogen is selected from the group consisting of hepatitis B, hepatitis C, and HIV.

22. The method of claim 19, wherein said needle-free valve is a positive displacement luer activated valve.

23. A method of reducing the potential for development of a air embolism during the placement of an indwelling catheter into the venous system of a patient comprising:
providing an indwelling catheter assembly comprising:
a needle-free valve assembly having a valve body, wherein said body comprises a first end and a second end, said body defines a passageway extending between said first and second ends, and said body defines a sealing surface in said passageway, and wherein said needle-free valve prevents air from being drawn into said catheter assembly;
a guidewire entry port defined by said needless valve assembly;
a length of catheter tubing affixed to said first end of said needle free valve; and
a stiffening member extending within said guidewire entry port and disposed within said needle-free valve, wherein said stiffening member extends through a portion of the length of catheter tubing;
introducing said indwelling catheter assembly into the patient's venous system; and
removing said stiffening member from said catheter assembly.

24. The method of claim 23, wherein said needle-free valve is a positive displacement luer activated valve.

25. The method of claim 23, wherein said indwelling catheter assembly further comprises a fitting permanently attached to said first end of said needle-free valve assembly to facilitate attachment of said catheter tubing.

26. The method of claim 23, wherein said catheter selected from the group consisting of a PICC, Midline, and Peripheral catheter.

27. A method of reducing the transmission of nosocomial infection during the aspiration of fluids from or delivery of fluids to an indwelling catheter device comprising:
providing an indwelling catheter assembly comprising:
a needle-free valve assembly having a valve body, wherein said body comprises a first end and a second end, said body defines a passageway extending between said first and second ends, and said body defines a sealing surface in said passageway;
a guidewire entry port extending through a portion of said needle-free valve assembly;
a length of catheter tubing affixed to said first end of said needle-free valve assembly; and
a stiffening member extending within said guidewire entry port and disposed within said needle-free valve, wherein said stiffening member extends through a portion of the length of catheter tubing;
introducing said indwelling catheter assembly into the patient's venous system;
removing said stiffening member from said catheter assembly;
swabbing said needle-free valve assembly with an antiseptic agent; and
drawing fluid from or delivering fluid through said needle-free valve.

28. The method of claim 27, wherein said nosocomial infection is selected from the group consisting of Staphylocooccus aureus, Candida spp., Escherichia coli, Pseudomonas aeruginosa, Acinetobacter calcoaceticus, and Klebsiella pneumoniae.

29. The method of claim 27, wherein said indwelling catheter is selected from the group consisting of a PICC, Midline, and Peripheral catheter.

30. The method of claim 27, wherein said needle-free valve is a positive displacement luer activated valve.

31. The method of claim 27, wherein said indwelling catheter assembly further comprises a fitting permanently attached to said first end of said needle-free valve assembly to facilitate attachment of said catheter tubing.

32. The method of claim 27, wherein said guidewire entry port is located on the surface of said valve body.

33. The method of claim 27, wherein said guidewire entry port is disposed within said fluid passageway of said valve assembly.

34. The method of claim 27, wherein said indwelling catheter assembly further comprises a detachable T connector.

35. The method of claim 27, wherein said indwelling catheter assembly further comprises a detachable Y connector.

36. A method of manufacturing an indwelling catheter assembly having a needle-free valve permanently attached thereto and a stiffening member, comprising:
providing a needle-free valve assembly having a valve body, wherein said body comprises a first end and a second end, said body defines a passageway extending between said first and second ends, and said body defines a sealing surface in said passageway;
locating a guidewire entry port on the surface of said valve body;
permanently attaching a length of catheter tubing to said first end of said needle-free valve assembly; and
extending a stiffening member through said guidewire entry port, past said needle-free valve, and into a portion of the interior of said catheter tubing.

37. The method of claim 36, further comprising removeably attaching a T connector to the second end of said valve body.

38. The method of claim 36, further comprising removeably attaching a Y connector to the second end of said valve body.

39. The method of claim 36, further comprising permanently affixing a fitting to said first end of said needle-free valve assembly to facilitate attachment of said catheter tubing or extension set.

40. The method of claim 36, wherein said catheter tubing is constructed from an elastomeric material selected from the group consisting of PolyEther Block Amide, PolyOlefins, PolyVinyl Chloride (PVC), PolyVinylidene Fluoride (PVDF), Styrene-Ethylene-Butylene-Styrene Block Copolymer, and Thermoplastic PolyUrethanes (TPUs).

41. The method of claim 40, wherein said polyolefins are selected from the group consisting of LDPE, LLDPE, HDPE, and PP.

42. The method of claim 36, wherein said fitting is constructed from molded plastic.

43. The method of claim 36, wherein said valve assembly is a positive displacement luer activated valve.

44. The method of claim 36, wherein said indwelling catheter is selected from the group consisting of a PICC, Midline, and Peripheral catheter.

45. An indwelling catheter assembly, comprising:
a needle-free valve assembly having a valve body, wherein said body comprises a first end and a second end, said body defines a passageway extending between said first and second ends, and said body defines a sealing surface in said passageway;
a stiffening means for increasing fluid flow area within said catheter assembly and increasing robustness of the catheter assembly;
means for extending or withdrawing said stiffening means within said catheter assembly;
catheter tubing; and
means for permanently attaching the first end of said needle-free valve assembly to said catheter tubing.
